(19) United States
(12) Patent Application Publication
(10) Pub. No.: US 2011/0165223 A1
(43) Pub. Date: Jul. 7, 2011

(54) Antitumor Immunization by Liposomal Delivery of Vaccine to the Spleen

(75) Inventors: George Sgouros, Ellicott City, MD (US); Hong Song, Reiserstown, MD (US); Mohana Lingappa, Baltimore, MD (US)

(73) Assignee: THE JOHNS HOPKINS UNIVERSITY, Baltimore, MD (US)

(21) Appl. No.: 12/811,620

(22) PCT Filed: Jan. 2, 2009
(86) PCT No.: PCT/US09/30039

§ 371 Date: Mar. 14, 2011

Related U.S. Application Data
(60) Provisional application No. 61/009,779, filed on Jan. 2, 2008.

(51) Int. Cl. A61K 9/127; A61K 39/00; A61K 39/12; A61K 47/46; A61P 35/00; A61P 37/04

(52) U.S. Cl. 424/450; 424/184.1; 424/277.1; 424/204.1; 424/278.1

(57) ABSTRACT

The present invention relates to methods for preventing, reducing or treating a variety of conditions, including cancer, and vaccines, compositions and liposomes used to elicit or amplify an immune response specific to the condition by delivering to the spleen of an individual a pegylated liposome construct having a diameter of greater than about 300 nm and including a therapeutic agent and an adjuvant for eliciting or amplifying the immune response.

The research resulting in the invention described herein was supported in part by Grant Numbers USAMRMC BC052595 and BC062968 from the U.S. Army. The U.S. Government has certain rights in the invention.

### BACKGROUND

Vaccine therapy is an attractive modality for treating conditions such as cancer because it is much less toxic and invasive than chemotherapy or surgery. However, immunotherapy in the form of anti-tumor vaccination has yielded occasional but not consistently promising results.

Cancer vaccine is a promising systemic therapy that activates host adaptive immunity to eradicate tumor cells. However, tumors induce several immune suppressive mechanisms to inhibit immune responses activated by some cancer vaccines. There is a need for vaccination strategies to overcome the body's immune tolerance of tumor cells. It is, therefore, desirable to enhance the anti-tumor immunity by overcoming tumor immunosuppression within the tumor microimmunoenvironment.

Delivery of vaccines using liposomes offers the potential advantage that all of the molecules involved in helping the body mount an immune reaction are packaged in a single delivery vehicle. Liposomal vaccines have been previously described, but these have generally been of the order of 100 nm in diameter and are formulated for blood circulation to target the individual tumors.

A fundamental problem in these vaccination methods is to present the target antigen to the appropriate population of cells (i.e., dendritic cells) so that the immune system will be activated sufficiently to mount an immune response against cells that exhibit the target antigen. The problem in many cases is that the immune system is not sufficiently activated and the immune response is of short duration or inadequate to eradicate the Ag+ cells. It is thought that this is, in part, because the most potent T-cell activating cell population is not itself activated by the vaccine. The problem is that to date, methods have not existed to deliver the vaccine directly to the most sensitive and potent T-cell activating cells.

In contrast, the present invention describes a liposomal construct having a diameter of greater than 300 nm that achieves a high concentration to the most sensitive and potent population of dendritic cells (i.e., the ones in the spleen). Delivery to this cell population is a function of the diameter of the liposomes.

The present invention relates to a vaccine delivery method that targets a therapeutic agent and adjuvant to the spleen, one of the most potent sites in the body for activating the immune system against the target antigen. The present invention provides for compositions, methods of use and methods of making a liposomal construct or formulation that can deliver the vaccine at high concentration to the spleen, thereby exposing the highly sensitive and potent immunostimulatory cells found in the spleen to the vaccine and associated adjuvant.

### BRIEF DESCRIPTION OF THE DRAWINGS

FIG. 1. Biodistribution of 650 nm (FIG. 1A), 400 (FIG. 1B) and 100 (FIG. 1C) diameter liposomes in mice at 1 and 6 hours after IV injection; 6 mice/group.

FIG. 2. Panel A and Panel B. Confocal microscope images (5 μm-thick) of liposomes (red) and FITC-labeled non-specific antibody (green) on spleen sections obtained 24 hrs after IV administration of rhodamine labeled liposomes.

FIG. 3. MicroSPECT/microCT imaging of liposome biodistribution (FIGS. 3A to 3M).

FIG. 4. Timeline for administration and dendritic cell uptake analysis.

FIG. 5. Dendritic cell uptake results of non-pegylated liposome constructs using flow cytometry by a magnetic separation technique.

FIG. 6. Dendritic cell uptake results of pegylated liposome constructs containing RNeu using flow cytometry by a magnetic separation technique.

FIG. 7. Depiction of the preparation of pegylated liposome constructs containing neu protein and GM-CSF.

FIG. 8. Time line for comparison study for PEG-Liposome with neu expressing NT₂ tumor lysate and GM-CSF versus control constructs having irradiated 3T3-newGM.

FIG. 9. Fluorescence Activated Cell Sorting (“FACS”) analysis of neu-specific IgG antibody (B-cell activation) in mouse serum using NT₂ cells and detected by secondary FITC-IgG₂ₐ antibody.

FIG. 10. Timeline of liposome-vaccine treatment protocol.

FIG. 11. Represents the tumor growth rate with and without liposome-vaccine after subcutaneous injection of 10⁶ NT₂ cells in FVB mice.

FIG. 12. Histogram representation of FACS analysis of neu-specific IgG antibody (B-cell activation) in mouse serum using NT₂ cells.

### DESCRIPTION

Described herein are vaccines, compositions and methods for the treatment of a variety of conditions. In particular, the present invention relates to methods for preventing, reducing or treating a variety of conditions, including cancer, and vaccines, compositions and liposomes used to elicit or amplify an immune response specific to the condition by delivering to the spleen of an individual a pegylated liposome construct having a diameter of greater than about 300 nm and including a therapeutic agent and an adjuvant for eliciting or amplifying the immune response.

In a particular embodiment, the present invention provides a composition comprising a pegylated liposome construct formulated for delivery to the spleen of an individual, wherein said liposome construct has a diameter of greater than about 300 nm and includes a therapeutic agent and an adjuvant for eliciting the immune response in said individual for preventing, reducing or treating a condition.

In another aspect, the invention provides a method for preventing, reducing or treating a condition comprising eliciting an immune response specific to said condition by delivering to the spleen of an individual a pegylated liposome construct, wherein said liposome has a diameter of greater than about 300 nm and comprises a therapeutic agent and an adjuvant for eliciting the immune response.

The individual may be a human or non-human mammal and the condition may be a disease or disorder and may include the presence of tumor cell or may be cancer. The tumor cell may be associated with a cancer, such as carcinomas of the gastrointestinal or colorectal tract, liver, pancreas, kidney, bladder, prostate, endometrium, ovary, testes, melanoma, dysplastic oral mucosa, invasive oral cancers, small cell and non-small cell lung carcinomas, breast cancer, hormone-dependent breast cancers, hormone independent breast cancers, transitional and squamous cell cancers, neurological malignancies, osteosarcomas, soft tissue sarcomas, hemangioamas, endocrinological tumors, hematologic neoplasias, carcinomas in situ, hyperplastic lesions, adenomas, fibromas, histiocytosis, chronic inflammatory proliferative diseases, vascular proliferative disease, and virus-induced proliferative diseases.

In a particular embodiment, the tumor cell may be associated with a proliferative disease such as leukemia, lymphoma, myeloproliferative disease, lymphoproliferative disease, neuroblastoma, glioma, and astrocytoma.

As used herein the “therapeutic agent” is an agent that when administered will prevent or alleviate a condition, disease or disorder with which a subject is afflicted or may be afflicted. The therapeutic agent may be an immunogen. For example, the condition can be cancer and the immunogen may be associated with a tumor cell from said cancer. The immunogen may be a tumor-specific antigen, molecule, peptide or protein, or a surface antigen specific to the tumor cell. The surface antigen may be included in a concentrated mixture or in a cell lysate derived from the tumor cell.

As used herein, the term “antigen” relates to any substance that elicits an immune response against the antigen in an animal, including a human, upon administration. An “immune response” may include a humoral and/or a cell-mediated immune response, which is accompanied by B cell proliferation and antibody secretion, activation of monocytes and/or macrophages as estimated by cytokine secretion (e.g. IL-1, IL-6, TNFα), activation and differentiation of dendritic cells (DC) as estimated by specific expression and/or up-or downregulation of specific surface antigens (e.g. MHC-class II, CD80, CD86, CD83, CD40, DC-LAMP which are upregulated and antigens, e.g. mannose-receptor, DEC-205, DC-SIGN which are downregulated) and by antigen-specific T cells, characterized by their expression of CD4 or CD8 and release of cytokines (e.g. IFNγ) upon activation (restimulation) with the appropriate antigen, in particular the same peptide antigen, used for immune response induction. The antigen may be tumor antigen, a viral antigen, a fungal antigen, a bacterial antigen, an autoantigen or an allergen.

As used herein, the term “tumor antigen” comprises all substances, which elicit an immune response against a tumor. Examples of tumor antigens include cancer-associated antigens belonging to gene products of mutated or recombined cellular genes, tumor virus antigens, overexpressed or tissue-specific differentiation antigens, and widely expressed antigens; or fragments or derivatives of any of the foregoing. Specific examples of a tumor antigen include cyclin-dependent kinase 4 (CDK4), p15^{Ink4b}, p53, AFP, β-catenin, caspase 8, p53, p21^{Ras} mutations, Bcr-abl fusion product, MUM-1 MUM-2, MUM-3, ELF2M, HSP70-2M, HST-2, KIAA0205, RAGE, myosin/m, 707-AP, CDC27/m, ETV6/AML, TEL/Aml1, Dekcain, LDLR/FUT, Pml-RARαTEL/AMLI, NY-ESO-1, members of the MAGE-family (MAGE-A1, MAGE-A2, MAGE-A3, MAGE-A4, MAGE-A6, MAGE-10, MAGE-12), BAGE, DAM-6, DAM-10, members of the GAGE-family (GAGE-1, GAGE-2, GAGE-3, GAGE-4, GAGE-5, GAGE-6, GAGE-7B, GAGE-8), NA-88A, CAG-3, RCC-associated antigen G250, human papilloma virus (HPV)-derived E6 E7 oncoproteins, Epstein Barr virus EBNA2-6, LMP-1, LMP-2, gp77, gp100, MART-1/Melan-A, p53, tyrosinase, tyrosinase-related protein (TRP-1 and TPR-2), PSA, PSM, MC1R, ART4, CAMEL, CEA, CypB, HER2/neu, hTERT, hTRT, iCE, Muc1, Muc2, PRAME RU1, RU2, SART-1, SART-2, SART-3, and WT1.

In a particular embodiment, the therapeutic agent is a neu related protein, peptide or antigen. The therapeutic agent may be a neu anti-breast cancer antigen derived from a human tumor cell. In one embodiment, the therapeutic agent is human HER2/Neu peptide (official symbol ErbB2; primary source HGNC:3430; organism Homo Sapiens). The therapeutic agent may be ErbB2/HER2/Neu as described in Jones, et al., Oncogene (1999) 18, 3481-3490. In yet another aspect, the therapeutic agent is a peptide antigen (RNEU₄₂₀₋₄₂₉) of rat HER2/neu.

As used herein, the term “viral antigen” includes any substance that elicits an immune response against a virus. Examples include Retroviridae, in particular HIV-1 and HIV-LP; Picornaviridae, in particular polio virus and hepatitis A virus; enterovirus, in particular human coxsackie virus, rhinovirus, echovirus; Calciviridae, in particular strains that cause gastroenteritis; Togaviridae, in particular equine encephalitis virus and rubella virus; Flaviridae, in particular dengue virus, encephalitis virus and yellow fever virus; Coronaviridae, in particular coronavirus; Rhabdoviridae, in particular vesicular stomatitis virus and rabies virus; Filoviridae, in particular Ebola virus or and Marburg virus; Paramyxoviridae, in particular parainfluenza virus, mumps virus, measles virus and respiratory syncytical virus; Orthomyxoviridae, in particular influenza virus; Bungaviridae, in particular Hantaan virus, bunga virus, phlebovirus and Nairo virus; Arena viridae, in particular hemorrhagic fever virus; Reoviridae, in particular reovirus, orbivirus and rotavirus; Birnaviridae; Hepadnaviridae, in particular Hepatitis B virus; Parvovirida, in particular parvovirus; Papovaviridae, in particular papilloma virus, simian virus-40 (SV40) and polyoma virus; Adenoviridae; Herpesviridae, in particular herpes simplex virus (HSV) 1 and 2, varicella zoster virus, cytomegalovirus (CMV), herpes virus; Poxyiridae, in particular variola virus, vaccinia virus and pox virus; and Iridoviridae, in particular African swine fever virus; Hepatitis C, and HPV L6, HPV L7, fragments and derivatives thereof.

As used herein, the term “fungal antigen” includes any substance that elicits an immune response against a fungus. Examples include Cryptococcus species, in particular Cryptococcus neoformans, Histoplasma species, in particular Histoplasma capsulatum, Coccidioides species, in particular Coccidioides immitis, Blastomyces species, in particular Blastomyces dermatitidis, Chlamydia species, in particular Chlamydia trachomatis, and Candida species, in particular Candida albicans.

As used herein, the term “bacterial antigen” includes any substance that elicits an immune response against a bacterium. Examples include Helicobacter species, in particular Helicobacter pyloris; Borelia species, in particular Borelia burgdorferi; Legionella species, in particular Legionella pneumophilia; Mycobacteria species, in particular M. tuberculosis, M. avium, M. intracellulare, M. kansasii, M. gordonae; Staphylococcus species, in particular Staphylococcus aureus; Neisseria species, in particular N. gonorrhoeae, N. meningitidis; Listeria species, in particular Listeria monocytogenes; Streptococcus species, in particular S. pyogenes, S. agalactiae; S. faecalis; S. bovis, S. pneumoniae; anaerobic Streptococcus species; pathogenic Campylobacter species; Enterococcus species; Haemophilus species, in particular Haemophilus influenzae; Bacillus species, in particular Bacillus anthracis; Corynebacterium species, in particular Corynebacterium diphtheriae; Erysipelothrix species, in particular Erysipelothrix rhusiopathiae; Clostridium species, in particular C. perfringens, C. tetani; Enterobacter species, in particular Enterobacter aerogenes, Klebsiella species, in particular Klebsiella pneumoniae, Pasturella species, in particular Pasturella multocida, Bacteroides species; Fusobacterium species, in particular Fusobacterium nucleatum; Streptobacillus species, in particular Streptobacillus moniliformis; Treponema species, in particular Treponema pertenue; Leptospira; pathogenic Escherichia species; and Actinomyces species, in particular Actinomyces israelli.

As used herein, the term “autoimmune antigen” includes any substance that elicits an immune response against a substance, e.g. a protein, which is normally present in the body, in particular in a healthy cell, tissue, or organ. Examples of autoimmune diseases include type 1 diabetes, conventional organ-specific autoimmune diseases, neurological diseases, rheumatic diseases, psoriasis, connective tissue diseases, autoimmune cytopenias, and other autoimmune diseases, or conventional organ specific autoimmunity, such as thyroiditis (Graves+Hashimoto's), gastritis, adrenalitis (Addison's), ovaritis, primary biliary cirrhosis, myasthenia gravis, gonadal failure, hypoparathyroidism, alopecia, malabsorption syndrome, pernicious anemia, hepatitis, anti-receptor antibody diseases and vitiligo, or neurological diseases such as schizophrenia, Alzheimer's disease, depression, hypopituitarism, diabetes insipidus, sicca syndrome and multiple sclerosis, or rheumatic diseases/connective tissue diseases such as rheumatoid arthritis, systemic lupus erythematous (SLE) or Lupus, scleroderma, polymyositis, inflammatory bowel disease, dermatomyositis, ulcerative colitis, Crohn's disease, vasculitis, psoriatic arthritis, exfoliative psoriatic dermatitis, pemphigus vulgaris, Sjogren's syndrome, or other autoimmune related diseases such as autoimmune uvoretinitis, glomerulonephritis, post myocardial infarction cardiotomy syndrome, pulmonary hemosiderosis, amyloidosis, sarcoidosis, aphthous stomatitis, and other immune related diseases.

As used herein, the term “allergen” includes any substance that elicits an immune response against other extraneous substance, not defined above. Examples may include pollen, such as from maple, birch, alder, hazelnut, mugwort, beach mountain cedar, oak, walnut, elm, olive, sycamore, cottonwood, white ash, and white pine; grass, such as from sweet vernal grass, orchard grass, Bermuda grass, oat grass, rye grass; insects, such as mites; food stuff, such as milk and milk products, nuts, such as peanuts, hazelnut and almonds; animal hair, such as hair derived from cat, horse, donkey, sheep, goat, dog, mice, rat, guinea pig, and rabbit.

The therapeutic agent may be located in the hydrophobic membrane of the liposome, the hydrophilic core of the liposome or on its surface.

As used herein, the term “adjuvant” is any substance that can be considered an activator of the immune system by stimulating receptors or pathways or both within cells of the immune system. Examples of adjuvants include unmethylated DNA comprising CpG dinucleotides (CpG motif); gel-like precipitates of aluminum hydroxide (alum); bacterial related proteins, peptides and products, e.g., from the outer membrane of Gram-negative bacteria; synthetic lipopeptide derivatives; peptidoglycan; zymosan; heat shock proteins (HSP); dsRNA and synthetic derivatives thereof; polycationic peptides; taxol; fibronectin; flagellin; imidazoquinoline; cytokines with adjuvant activity; Tween 80 and Span 85 (sorbitan-trioleate) and QS-21, a more highly purified derivative of Quil A, non-ionic block polymers, saponins and derivatives thereof; polyphosphazene; N-(2-Deoxy-2-L-leucylamino-.beta.-D-glucopyranosyl)-N-octadecyldodecanoyl-amide hydroacetate (BAY R1005), 25-dihydroxyvitamin D3 (calcitriol); DHEA; murametide [MDP(Gln)-OMe]; murapalmitine; polymers of lactic and/or glycolic acid; polymethyl methacrylate; sorbitan trioleate; squalane; stearyl tyrosine; squalene; theramide, and synthetic oligopeptides. Specific examples include CpG ODN with phosphorothioate (PTO) backbone (CpG PTO ODN) or phosphodiester (PO) backbone (CpG PO ODN); monophosphoryl lipid A (MPLA), lipopolysaccharides (LPS), muramyl dipeptides and derivatives thereof; Pam.sub.3Cys; HSP 70; Poly I:poly C; poly-L-arginine; GM-CSF, interleukin-(IL-)2, IL-6, IL-7, IL-18, type I and II, interferons, interferon-gamma, TNF-alpha; MF59 consisting of squalene; Poloxamer 401, immunostimulatory fragments from saponins; and MHCII-presented peptides.

In a particular embodiment, the adjuvant may be immunostimulatory and can elicit or amplify an immune response or reaction. In a certain aspect, the adjuvant may be a cytokine or an agent that stimulates cytokine receptors, such as GM-CSF.

Examples of cytokines include a lymphokine, interleukin (IL) or chemokine. A cytokine may elicit a specific effect in promoting proliferation of T-cells that cause cytotoxic effects and stimulates cytokine receptors such as those from the Immunoglobulin (Ig) superfamily, Haemopoietic Growth Factor (type 1) family, Interferon (type 2) family, Tumor necrosis factors (TNF) (type 3) family, and Seven transmembrane helix family. A cytokine may be from the IL-1 family, such as IL-1 and IL-18, the IL-17 family, the IL-2 subfamily, the interferon (IFN) subfamily, or the IL-10 subfamily. A cytokine may be GM-CSF, IL-2, IL-4, IL-6, IL-7, IL-8, IL-10, IL-13, IFN-α, TGF-β, IFN-γ.

The adjuvant may be located in the hydrophobic membrane of the liposome, the hydrophilic core of the liposome or on its surface.

A liposome may be made in a variety of manners and the size of the diameter may be characterized and prepared in a number of ways, e.g., as described in Lasic, “Liposomes in Gene Delivery,” Biophysical Journal (April 1998) V. 74, 2138-2139, and Castile, et al., “Factors affecting the size distribution of liposomes produced by freeze-thaw extrusion,” International Journal of Pharmaceutics 188 (1999) 87-95. The diameter of the liposomes can be controlled, e.g., by extrusion of the liposomal composition through sieves or meshes with a known pore size, e.g., as described in Mayhew et al. (1984) Biochim. Biophys. Acta 775:169-174 or Olson et al. (1979) Biochim. Biophys. Acta 557:9-23. A specific size provided herein indicates an average/mean value.

The liposome construct of the present invention may have a diameter of greater than 300, or from about 300 to about 1000 nm, from about 400 to about 900 nm, from about 500 to about 800 nm, from about 600 to about 700 nm, from about 700 to about 800 nm, from about 600 to about 650 nm, from about 650 to about 700 nm, or of about 600, 650, 700, 750, or 800 nm, or a range within these sizes. In a particular embodiment, the liposome has a diameter of about 650 nm.

The present invention may further include a chemical moiety attached to the membrane of the liposome. The chemical moiety may be for targeting, stabilizing or protecting the liposome construct. As used herein the term “attached” relates to a direct or indirect, covalent or non-covalent bond and connection, respectively, between a chemical moiety and another component of the liposome. Examples of chemical moiety include biotin-streptavidin, amino-reactive groups (e.g. carbodiimides, hydroxylmethylphosphine, imidoester, N-hydroxysuccinimide esters, isothiocyanates, isocyanates), sulfhydryl-reactive groups (e.g. maleimides, haloacetyls, pyridyl disulfides, aziridines) carboxyl-reactive molecules (e.g. carbodiimides, carbodiimidazole, diaoalkanes), hydroxyl-reactive groups (e.g. carbonyldiimidazole, alkyl halogens, isocyanates), or can include a stabilizing moiety for increasing the circulation time of the liposome once it is administered, such as ganglioside GM1, phosphatidylinositol or polyethylene glycol (PEG), e.g., PEGs having a molecular mass between about 1,000 and about 10,000 g/mol. Targeting moieties may also include detergents, proteins, and peptides, such as an antibody or fragment thereof, a single-chain antibody or fragment thereof, a receptor ligand or fragment thereof; a carbohydrate; or a ligand. Specific examples may include natural or synthetic receptor-binding peptides and mimetics thereof, mono- or oligosaccharides, receptor ligands or fragments thereof, antibodies or fragments thereof, all of which are directed against DC-specific surface molecules or receptors, in particular CD54 (ICAM-1) and ICAM-2, mannose receptor, CD207 (langerin), ASGPR, CLEC-1, CLEC-2, DCIR, dectin-1, DC-SIGN, DEC-205, BDCA-2, TLR-1, TLR-2, TLR-3, TLR-4, TLR-5, TLR-7, TLR-9, CD40, CD16/32 (FcγR-III and -II), CD11, CD1a, CD1d, and MHC class II.

In a particular embodiment, the liposome construct may include a polyethylene glycol (PEG) moiety with a molecular mass of 1000 to 10000 g/mol. In one aspect, the PEG is PEG 2000 (molecular weight of 2,000 g/mol.). A chemical moiety may be included in an amount of between about 1 to about 20 mol % of the components of the liposomal membrane. In particular aspects, the chemical moiety is in an amount of between about 3 to about 10 mol % or between about 3 to about 4 mol %.

In another aspect, the chemical moiety may be receptors that help activate the immune response, receptors against dendritic cells, CD4 helper or CD8 T cells.

As used herein, the terms “treatment,” “treating,” etc., refer to obtaining a desired pharmacologic and/or physiologic effect. The effect may be prophylactic in terms of completely or partially preventing a condition or disease or symptom thereof and/or may be therapeutic in terms of a partial or complete cure for a condition or disease and/or any adverse affect attributable to the condition or disease. “Treatment,” thus, for example, covers: (a) preventing the condition or disease from occurring in an individual who is predisposed to the condition or disease but has not yet been diagnosed as having it; (b) inhibiting the condition or disease, such as, arresting its development; and (c) relieving, alleviating or ameliorating the condition or disease, such as, for example, causing regression of the condition or disease.

The liposome construct may be administered intravenously to the individual. The liposome may be administered in other manners as well, e.g., intraparenterally, intramuscularly, or subcutaneously, so long as the construct ends up in the spleen. The therapeutic vaccine can be formulated into preparations for injection by dissolving, suspending or emulsifying them in an aqueous or non-aqueous solvent, such as vegetable or other similar oils, including corn oil, castor oil, synthetic aliphatic acid glycerides, esters of higher aliphatic acids or propylene glycol; and if desired, with conventional additives such as solubilizers, isotonic agents, suspending agents, emulsifying agents, stabilizers and preservatives so long as such preparations do not compromise the liposomal structure.

The term “unit dosage form,” as used herein, refers to physically discrete units suitable as unitary dosages, each unit including a predetermined quantity of compounds of the present invention calculated in an amount sufficient to produce the desired effect in association with a pharmaceutically acceptable diluent, carrier or vehicle. The specifications for the novel unit dosage forms of the present invention depend on the vaccine employed and the effect to be achieved, and the pharmacodynamics associated with each vaccine in the patient.

The appropriate dose to be administered depends on the subject to be treated, such as the general health of the subject, the age of the subject, the state of the disease or condition, the weight of the subject, etc. The vaccine can be administered in a single or, more typically, multiple doses. They may be formulated together into a single composition, or administered separately, either simultaneously or at different times. Preferred dosages for a given compound are readily determinable by those of skill in the art by a variety of means. Other effective dosages can be readily determined by one of ordinary skill in the art through routine trials establishing dose response curves. The amount of vaccine to be administered will, of course, vary depending upon the particular compound.

The liposome construct may be administered to a human in a dose of an effective amount of from about 1.0×10¹⁵ to about 1.0×10²⁰ liposomes/kilogram of body weight. The dosage amount may depend on the size of the liposome, e.g., for 1000 nm diameter liposomes the ideal dose may be 0.5×10¹⁸ liposomes per kg/body weight in a human, while for 300 nm diameter liposomes, the ideal dose may be 19×10¹⁸ liposomes per kg/body weight in a human. In another aspect, the liposome construct may be administered to a human in a dose of an effective amount of from about 4.7 to about 15 nanomoles of liposome concentration/kilogram of body weight.

In another exemplary aspect, for a 20 mg mammal (e.g., a mouse), the liposome construct for intravenous injection may be 0.3 nmoles liposome concentration, 1.93×10¹⁴ number of liposome molecules for 300 nm diameter liposome; 0.1464 nmoles liposome concentration, 8.80×10¹³ number of liposome molecules for 650 nm diameter liposome; or 0.094 nmoles liposome concentration, 5.60×10¹³ number of liposome molecules for 1000 nm diameter liposome.

The frequency of administration of the vaccine, as with the doses, will be determined by the medical practitioner based on age, weight, disease status, health status and patient responsiveness. Thus, the vaccine may be administered one or more times daily, weekly, monthly or as appropriate as conventionally determined. The vaccine may be administered intermittently, such as for a period of days, weeks or months, then not again until some time has passed, such as 3 or 6 months, and then administered again for a period of days, weeks, or months. In a particular aspect, the vaccine or liposome construct may be administered to a human as a one-time dose or daily, weekly, every two weeks, or every month.

The therapeutic agent may be contained in a cell lysate or provided in a concentrate mixture. In one aspect of the invention, the therapeutic agent is contained in a cell lysate and provided in an amount from about 1 to 20 mg/ml per dose. In another aspect, the therapeutic agent is contained in a cell lysate and provided in an amount of about 4 mg/ml per dose. In another embodiment, the therapeutic agent is prepared for a 20 g mammal (mouse) in an amount from about 100 μg to 1000 μg of lysate per dose or about 800 μg of lysate with liposome of 0.3 nmoles per dose. In another embodiment, the therapeutic agent is provided in a concentrated form/mixture in an amount from about 1.0 to 1000 mg/ml per dose.

The therapeutic agent may be in an amount of from about 3 to 30% w/w of the liposome construct.

The adjuvant may be provided in an amount from about 0.01 to 2.0 mg/ml per dose. In another aspect, the adjuvant is provided in an amount of about 0.05 mg/ml per dose. In another embodiment, the adjuvant, GM-CSF, is prepared for a 20 g mammal (mouse) in an amount from about 0.1 μg to 10 μg or 1 μg (3-9×10⁵ units) with liposome of 0.3 nmoles per dose.

The adjuvant may be in an amount of from about 0.0029 to 0.29% w/w of the liposome construct.

In another aspect, a liposome construct can be prepared for a 20 g mammal (mouse) containing an amount of about 0.1 μg to about 10 μg of GM-CSF and about 100 μg to 1000 μg of lysate containing therapeutic agent, per dose. As a specific example, a construct can be prepared for a 20 g mammal (mouse) containing an amount of about 1 μg (3-9×10⁵ units) of GM CSF and about 800 μg of lysate containing therapeutic agent, per dose.

As mentioned above, effective amounts of the vaccine are administered to an individual, where “effective amount” means a dosage sufficient to produce a desired result. The liposome construct may be administered in an effective amount to provide sufficient activation of the individual's immune system, generation of antibody or eradicate of antigen expressing (Ag+) cells.

In a particular aspect, the liposome construct causes an increase of the immune response at least by 5%, at least by 10%, at least by 20%, at least by 50%, at least by 100%, at least by 200%, or at least by 1000%. The response may be measured in a variety of ways, e.g., by measuring antibody production in response to a specific antigen or by T cell activation. The antibody increase may be by at least 5% or at least 100 fold.

In another aspect, the liposome construct is administered to treat cancer and causes a cessation in tumor growth, a decrease of tumor size or growth delay or eradication of tumor cells. An “effective amount” of reduction in tumor size may be at least by 5%, 10%, at least by 20%, at least by 50%, at least by 90%, at least by 100%. In yet a further aspect, the tumor growth delay is of at least one week to about five weeks.

The liposome construct may be formulated to release its content or activate without rupturing. In a certain embodiment, when the liposome construct reaches the spleen, it is exposed to an external stimulus, e.g., heat or external beam radiation, so that the therapeutic agent and adjuvant are released.

The administered liposome construct is formulated so that it localizes to the spleen rapidly and at very high concentration. In one aspect, the construct is localized to the spleen within one hour after administration. In another aspect, the liposome construct is localized to the spleen in an amount of greater than 100% ID/gm. after administration.

The administered liposome construct is formulated so that it is directed to the periarteriolar lymphoid sheath (PALS) contained within the white pulp (WP) region of the spleen. In another aspect, the administered liposome activates host adaptive immunity and recruits cytotoxic T lymphocytes (CTL) to eradicate the tumor cells. In yet another embodiment, the liposome activates naïve T-lymphocytes, antigen-presenting cells (APCs) and interdigitating (reticulum) cells (IDCs) that are derived from circulating dendritic cells in the spleen and overcomes an individual's immune tolerance of the tumor cells.

In one embodiment, the liposome construct may include an antigenic human or rat HER2/neu epitope, e.g., RNEU₄₂₀₋₄₂₉ on the exterior surface of the liposome construct or in the interior (core) and GM-CSF on the exterior surface of the liposome construct or in the interior (core). The liposome construct may further comprise CpG oligonucleotides on the exterior surface or in the interior (core). The construct may be formulated with the therapeutic agent, e.g., human or rat neu epitope, which is contained in a cell lysate, e.g., of NT2 cells, and GM-CSF.

As used herein, the singular forms “a”, “an”, and “the” include plural forms unless the context clearly dictates otherwise. Thus, for example, reference to “a liposome” includes a plurality of such liposomes.

Kits with multiple or unit doses of the vaccine, are included in the present invention. Such kits, in addition to the containers containing the multiple or unit doses of the vaccine, optionally include an informational package insert with instructions describing the use and attendant benefits of the vaccine components in treating the diseases/conditions.

As will be evident to those of skill in the art, the compositions and methods described herein can also be used with vaccines used to treat opportunistic infections and the like which occur frequently in cancer patients.

The invention is to be understood as not being limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All publications mentioned herein, including patents, patent applications, and journal articles are incorporated herein by reference in their entireties including the references cited therein, which are also incorporated herein by reference.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

This application claims priority to U.S. provisional application No. 61/009,779, filed Jan. 2, 2007, which is hereby incorporated herein by reference.

Abbreviations: CD4=protein co-receptor expressed mostly on the surface of helper T cells. CD8=protein co-receptor expressed on the surface of cytotoxic T cells. CpG=regions of DNA (stands for cytosine and guanine separated by a phosphate). eggPC=egg Phosphatidylcholine. FITC=fluorescein isothiocyanate. GM-CSF=granulocyte-macrophage colony-stimulating factor. HER2=Human EGF Receptor 2, a protein that is overexpressed on the surface of some breast cancer cells. ICLV=Intracardiac Left Ventricle injection used to inoculate breast cancer metastasis in bone and liver. ICS=intracellular cytokine staining. In-111=Indium-111, radionuclide with a 2.8 day half-life. IV=intravenous. MHC=The major histocompatibility complex expressed on the surface of cells. MTD=Maximum Tolerated Dose. Neu-N=designation of transgenic mice that express the wild-type (N), rat version, of the HER2/neu receptor. NT-2=a mouse tumor cell line, derived from Neu-N mice that expresses the wild type rat neu receptor. RNEU=immunodominant rat HER-2/neu epitope. PBS=phosphate buffered saline. PEG=Polyethylene glycol (polymer). PET=positron emission tomography that detects molecules labeled with positron (anti-electron)-emitting radionuclide. sub-Q=subcutaneous.

### EXAMPLES

### Example 1

### Biodistribution Studies

The biodistribution of ¹¹¹In-loaded liposomes was evaluated in 4-6 week old CD1 mice. Six mice per group were injected via the tail vein with polyethylene glycol (PEG)-coated eggPC/Chol(1:1) liposomes of 100, 400 or 650 (nominal) diameter. Liposome diameters were obtained by dynamic light scattering as described in Banchereau, et al., 1998, Nature 392, 245-252.

Liposomes of different diameters containing 2 mM Diethylene triamine pentaacetic acid (DTPA) were produced and purified with size exclusion Sephadex G-50 column to remove free DTPA before In-111 loading. 200 μCi In-111 was loaded into Approx. 3.2×10¹² liposomes in the presence of oxine. The loading efficiency was 79.1%±7.0%. Liposomes were purified again with Sephadex G-50 column to remove any remaining free In-111. For each size of liposome, 2.0×10¹¹ liposome were injected via the tail vein. At 1 and 6 hrs after injection, the mice were lightly anesthetized and sacrificed by cervical dislocation. After sacrifice, the blood, heart, lungs, liver, spleen, kidneys, stomach, intestines, muscle and bone were collected, weighted and counted for radioactivity using a gamma counter (LKB, Wallac). An additional experiment was performed to obtain a 24 hr time-point for the 650 nm diameter liposomes.

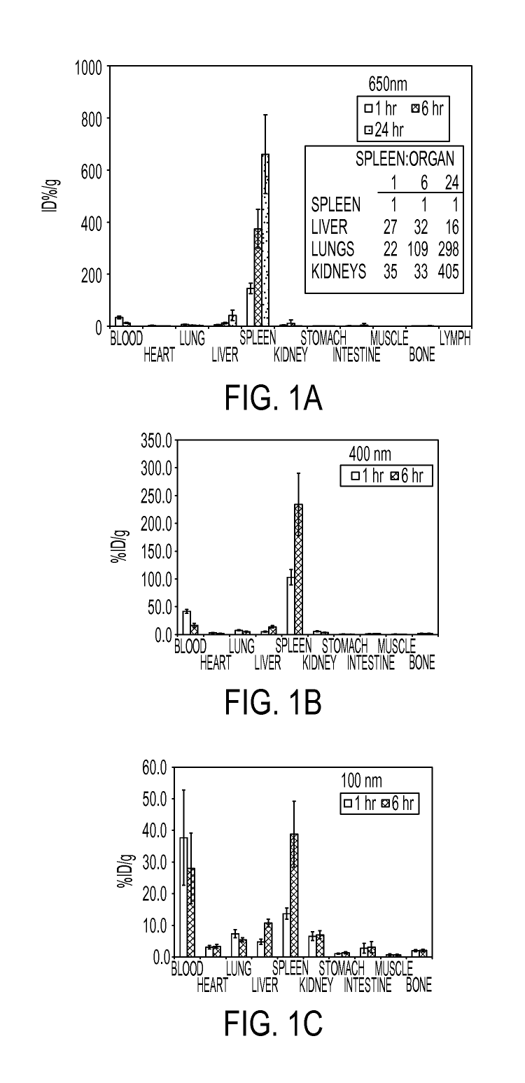

FIG. 1A shows the biodistribution of 650 nm diameter liposomes in mice at 1, 6 and 24 hours after IV injection. The inset in FIG. 1A provides the spleen organ activity concentration ratio showing the high relative localization of the liposomes to the spleen at the different time-points. FIGS. 1B and 1C show the biodistribution of 400 nm and 100 nm, respectively, diameter liposomes in mice at 1 and 6 hours after IV injection. At 650 nm, a very rapid sequestration to spleen is shown resulting in spleen:liver and spleen:lung of ˜30 as early as 1 hr PI and lasting to at least 6 hr PI.

The large, 650 nm-diameter PEGylated-liposomes, injected IV, localized to the spleen rapidly and at very high concentration showing that splenic localization is highly dependent upon the size distribution of the liposomes with smaller liposomes (100 and 400 nm diameter) not showing the level of localization and retention seen with larger liposomes.

### Example 2

### Confocal Microscopy of Liposome Distribution

Liposomal microdistribution relative to splenic macrophages was evaluated. Pegylated eggPC/Chol(1:1) liposomes with size of 650 nm were purified with size exclusion Sephadex G-50 column. Liposomes were labeled with rhodamine for fluorescent imaging. Two female FVB mice were injected with 140 μl of the liposomes via tail vein. 24 hr post injection, they were sacrificed and the spleens were dissected rapidly and snap frozen in liquid nitrogen. The spleen was then mounted on OCT medium and serially sectioned at 20 μm thickness on a cryomicrotome. The cut section was fixed with 3.7% formaldehyde at room temperature for 20 mins followed by permeabilization with 0.1% saponin for 10 mins. The slides were dip washed several times in PBS. To label macrophages in the spleens, each slide was incubated with 40 μl 2 μg/ml FITC conjugated rat anti-mouse F4/80 antibody for 1 hr at room temperature under dark condition. Parafilm was used to cover the slides to ensure uniform antibody distribution and prevent evaporation. After incubation, the slides were dip washed in PBS several times and dried overnight under dark conditions. The slides were then examined by confocal microscopy.

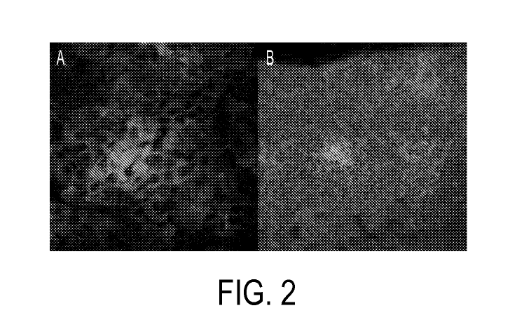

FIG. 2, panel A with (100×) liposomes only (slide not incubated w/antibody) and panel B with (40×) liposomes (antibody), shows confocal microscope images (5 μm-thick) of liposomes (red) and FITC-labeled non-specific antibody (green) on spleen sections obtained 24 hrs after IV administration of rhodamine labeled liposomes.

FIG. 2, Panel A depicts the cell-level distribution of liposomes. Liposomes were seen both outside cells and also intracellularly, this was confirmed by examining multiple CM slices through the sample. Panel B depicts a spleen section at lower magnification that has been counterstained with a non-specific FITC-labeled antibody. The pattern of rhodamine fluorescence, reflecting the distribution of liposomes is consistent with liposome localization in the white pulp region of the spleen.

The white pulp region is T-cell rich and also contains a high concentration of (interdigitating) dendritic cells. The architecture and nature of the cells present in this region are optimized for the processing and presentation of antigens for activation of naïve T-lymphocytes as described in Stein, et al., 1980, J Histochem. Cytochem. 28 August, (8), 746-60; and Dijkstra, et al., 1982, J. Reticuloendothel Soc. September, 32(3), 167-78. (5).

The observation that the periarteriolar lymphoid sheath (PALS), contained within the white pulp (WP) region of the spleen, contains T-cells, together with antigen-presenting cells (APCs) and also interdigitating (reticulum) cells (IDCs) that are derived from circulating dendritic cells. The IDCs have long, membrane processes and are strongly major histocompatibility complex (MHC) class II (MHC-II)-positive (MHC II is necessary for antigen presentation to T-cells). They also express high levels of co-stimulatory molecules, such as B7. These regions are the most potent cells for the processing and presentation of antigens for activation of naïve T-lymphocytes.

### Example 3

### Imaging, In Vivo PEG and Non-PEG Liposomes at 650 nm Diameter

The biodistribution of PEG and non-PEG liposomes at 650 nm diameter was examined by combined μSPECT/μCT imaging (Gamma-Medica XSPECT fitted with a custom-made pinhole collimator) at 1, 6, 24, 48, and 72 h post tail vein injection of 200 μCi ¹¹¹In-loaded liposomes in 100 μl PBS. Mice were anesthetized by isofluorane inhalation. The SPECT image acquisition parameters were 64 angles at 45 sec per angle, dual heads, with a 5.7 cm field of view and a 4.3 cm radius of rotation. MicroCT imaging was performed immediately after the end of the SPECT study without moving the animal. Images were reconstructed using an iterative reconstruction algorithm included in the Gamma-Medica software.

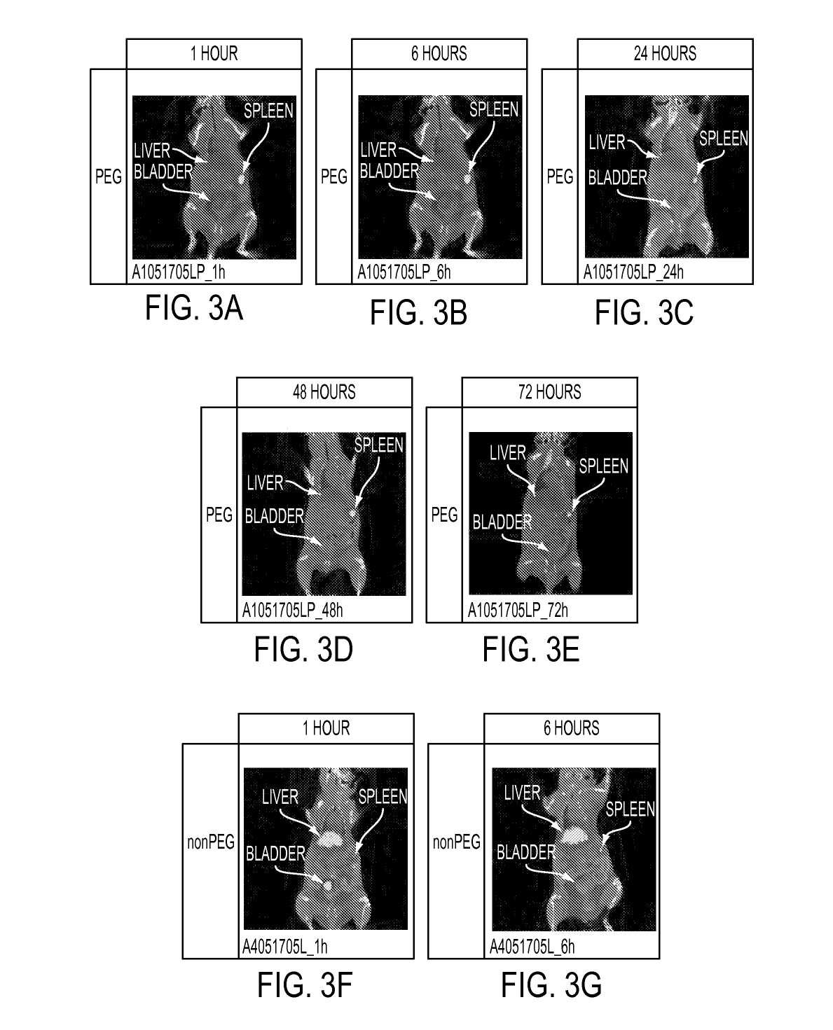

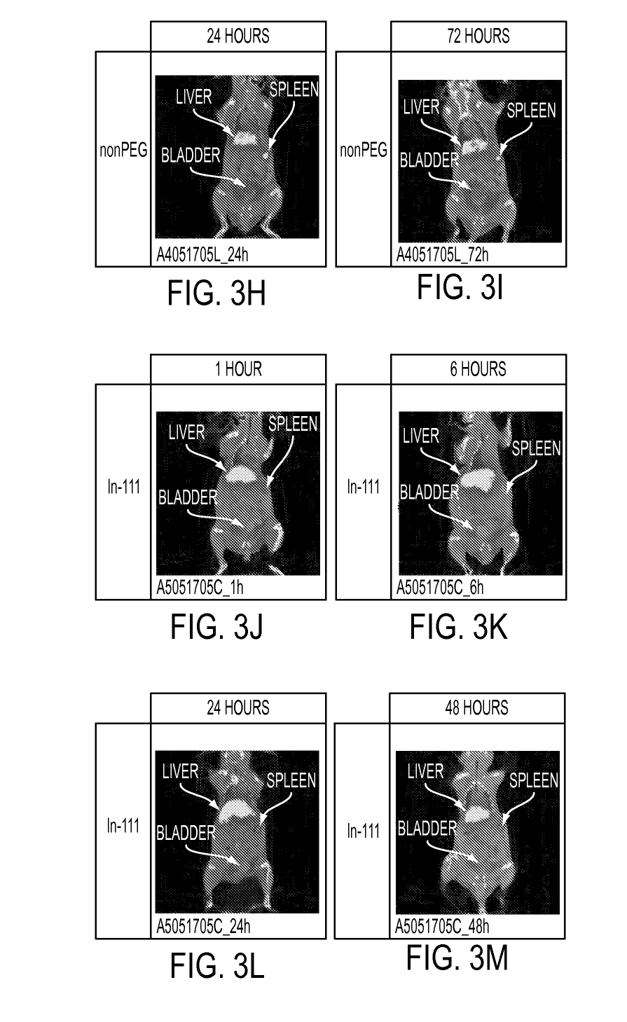

Results are presented in Figure set 3, FIGS. 3A to 3M, showing coronal μCT images with OPECT images of ¹¹¹In superimposed. Top row FIGS. 3A to 3E show ¹¹¹In-loaded 650 nm diameter PEG liposomes, middle row FIGS. 3F to 3I show ¹¹¹In-loaded 650 nm diameter nonPEG liposomes and bottom row FIGS. 3J to 3m have free ¹¹¹In. A linear OPECT “hot metal” scale (white−=high activity) was used for all images; in all cases max intensity was set to 49% of max pixel value.

### Example 4

### Dendritic Cell Uptake of Pegylated Liposome Constructs Containing RNeu

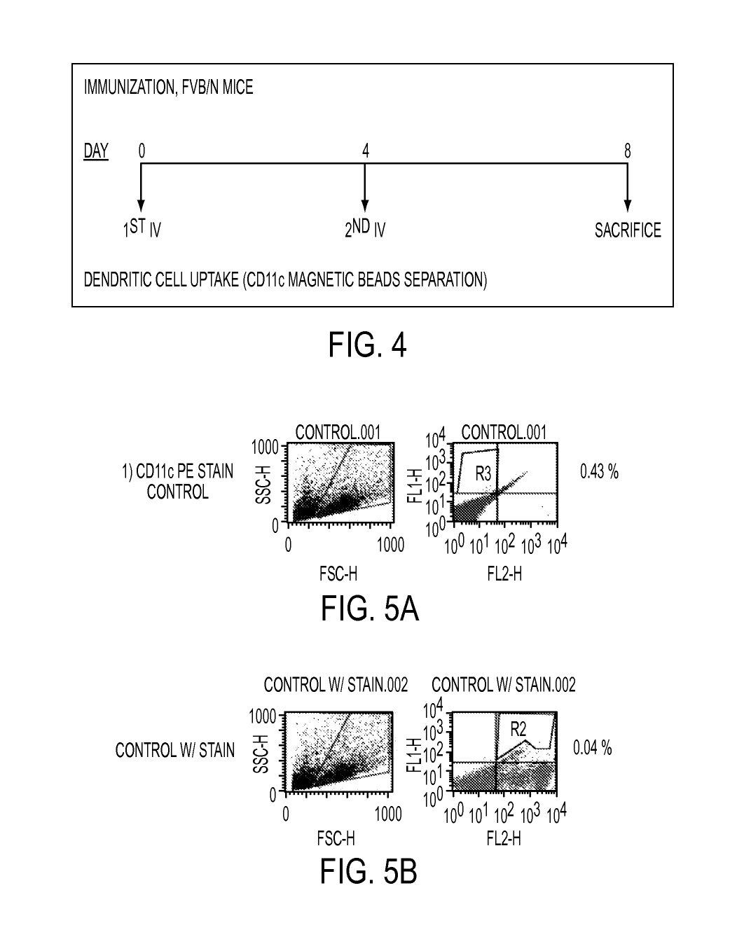

Liposomal constructs were prepared and examined for dendritic cell uptake following IV administration. The various liposomal constructs were stained with FITC for flow cytometry detection. The different liposomes were administered at day zero and 4 and the mouse was sacrificed 4 days after the last injection at day 8 (FIG. 4), the spleen was extracted rapidly and the cells dissociated. Dendritic cells were isolated for flow cytometry by a magnetic separation technique. Results are shown in Figure sets 5 and 6.

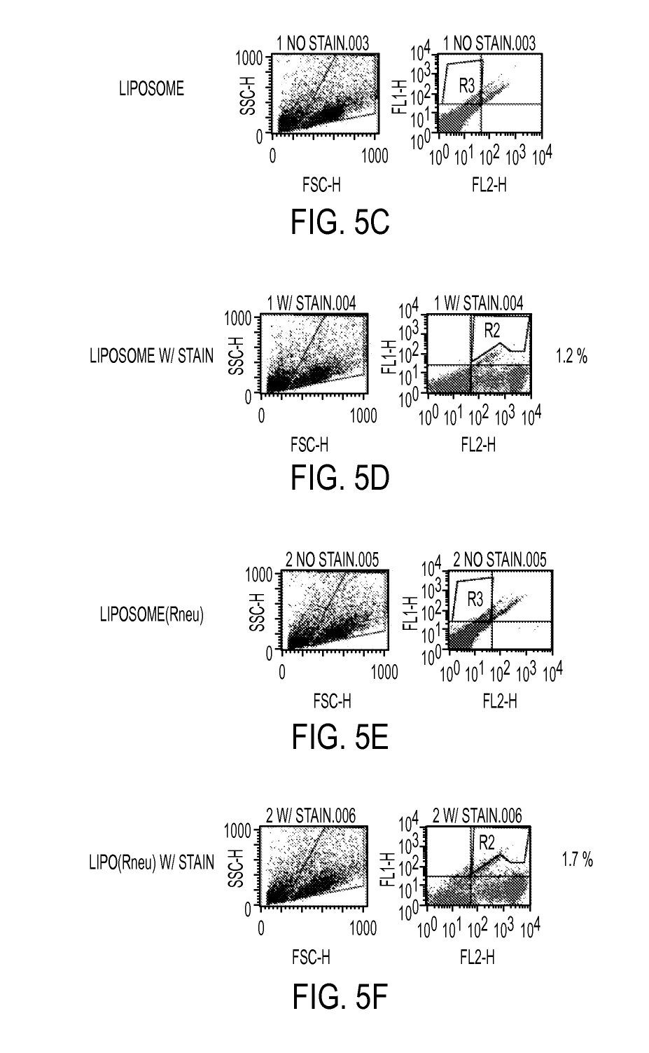

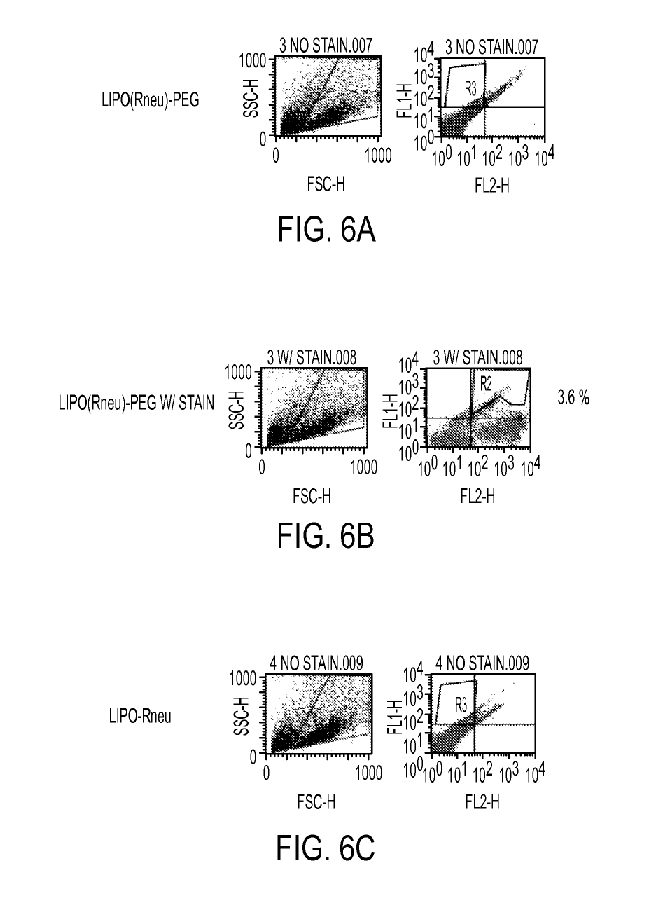

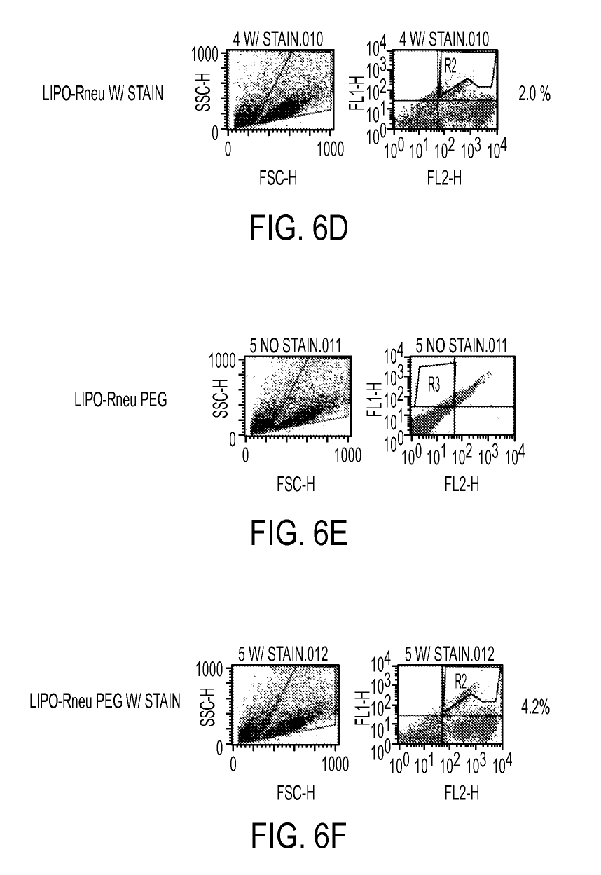

Dendritic cell uptake of non-PEG liposomes with or without the immunostimulatory peptide RNeu was less than 2%. Dendritic cell uptake of PEG liposomes was approximately 4%. No difference was observed for PEG constructs with RNeu in the hydrophilic interior (FIGS. 5E, 5F, 6A, 6B) of the liposomes versus placing the peptide on the surface of the PEGylated liposome (FIGS. 6C, 6D, 6E 6F).

### Example 5

### Methods for Preparing and Analyzing Liposomal Constructs

A variety of methods may be used to prepare large pegylated liposomes by the extended hydration method as described in Banchereau, et al., 1998, Nature 392, 245-252. RNEU₄₂₀₋₄₂₉ may be used with GM-CSF and can be passively loaded into the hydrophilic interior of the liposome. RNEU₄₂₀₋₄₂₉ has been shown to elicit activation of T-cells as described in Ercolini, et al., 2005, J Exp. Med. 16;201(10):1591-602.

Biodistribution and imaging studies can be performed using the various liposome vaccine constructs passively loaded with In-111 and imaged by microSPECT/CT. Using quantitative biodistribution is obtained by sacrificing the animals and extracting tissues for gamma counting. Microdistribution is obtained by optical, fluorescent microscopy/imaging, ex vivo of fluorescein-tagged liposome vaccine constructs.

### Example 6

### Efficacy Studies

Efficacy studies can be performed using the neu-N transgenic mouse model as described in Song, et al. Cancer Res 2008 May 15; 68(10):3873-80 and Song, et al. Clin Cancer Res 2008 Oct. 1; 14(19):6116-24. Efficacy studies are performed in mice with orthotopic and metastatic breast tumors using the administration dose. Treatment is initiated 3 days after fatpad tumor inoculation and 5 days post ICLV injection. Orthotopic tumors are generated in 20-25 g neu-N mice (Taconic) by injecting 5×10⁴ NT2 cells in the fatpad of the #3 mammary gland. Metastases to bone and liver are established by injecting 10⁵ NT2 cells in 0.1 ml PBS into the left cardiac ventricle, 1 mm below the clavicle and 5 mm to the left of the midline between the 2^{nd} and 3^{rd} intercostals, using a 1 ml syringe fitted with a 26G ½″ needle. Successful injections are denoted by the push of bright red arterial blood into the syringe tip. Approximately 85% of 9 successful injections (bright red blood at syringe tip) lead to confirmed bone and liver metastases.

### Example 7

### Preparation of Pegylated Liposome Constructs Including Neu Protein and GM-CSF

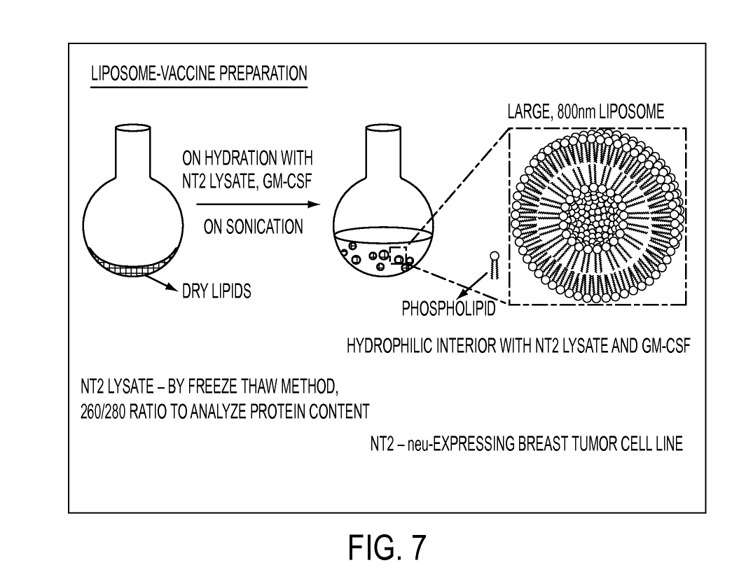

Liposome constructs were prepared by hydration of dry lipids (PC:Cholesterol, 3-4% DSPE-PEG2000) with neu expressing NT₂ tumor lysate and GM-CSF followed by bath sonication or freeze thaw and then extrusion to narrow down the liposome size. Tumor lysate was prepared by 6-7 cycles of freeze thaw of NT₂ cells followed by centrifugation at 15000 g for 1 hour and then passed through 0.22 μm filter membrane. This is depicted in FIG. 7.

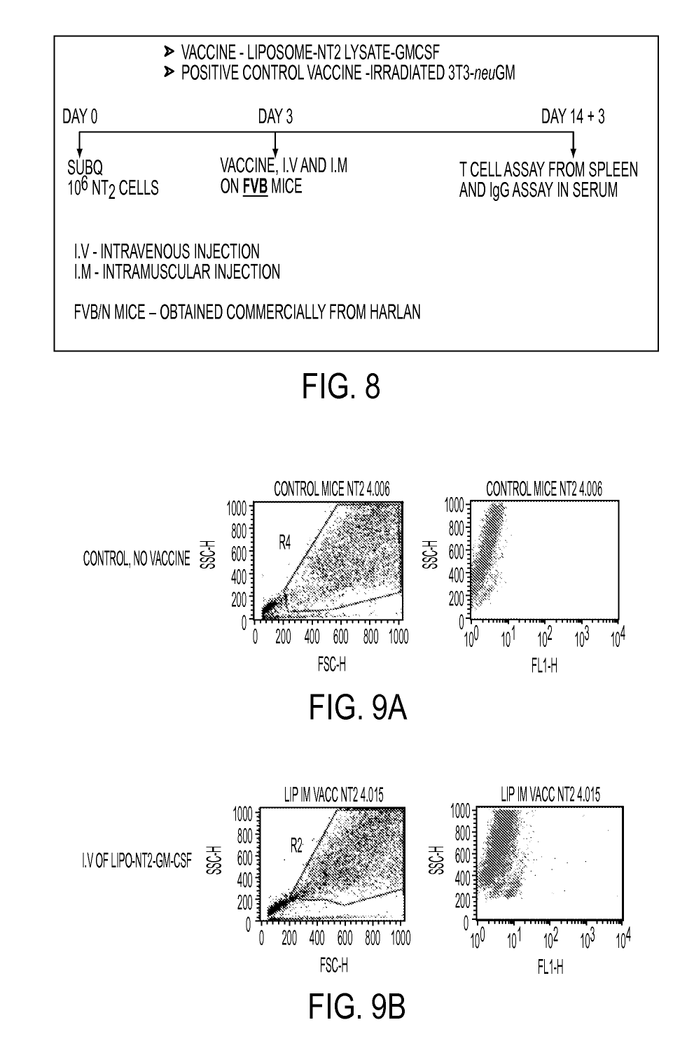

Positive control constructs were prepared using irradiated 3T3-newGM for comparison studies, FIG. 8.

### Example 8

Preparation of Liposome Constructs Including lysate and GM-CSF

The following constructs were prepared for a single intravenous injection for mice with 20 g body weight: 1 μg (3-9×10⁵ Units) of GM CSF with lysate of 800 μg of 0.3 nmoles per dose per mouse.

| Size, nm | Liposome conc., nmoles | # of liposomes |
|---|---|---|
| 300 | 0.3 | 1.93E+14 |
| 650 | 0.1464 | 8.80E+13 |
| 1000 | 0.094 | 5.60E+13 |

### Example 9

### FACS Analysis of Neu-Specific IgG Antibody

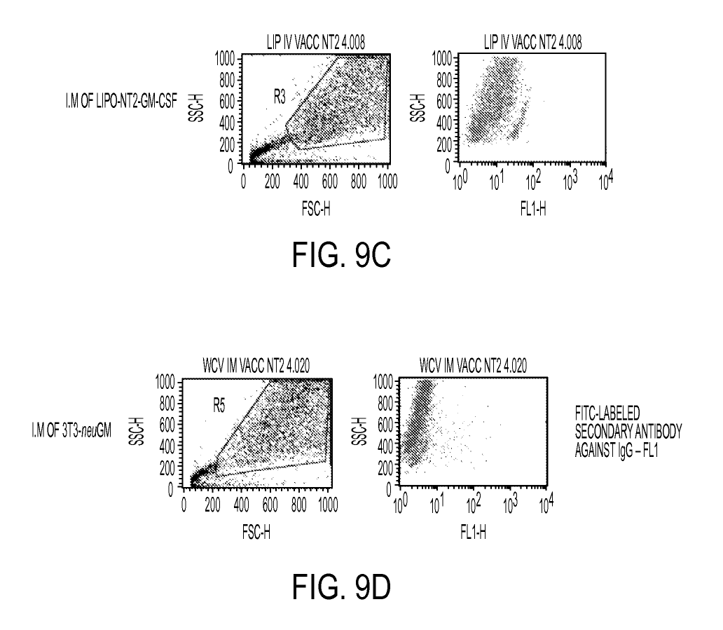

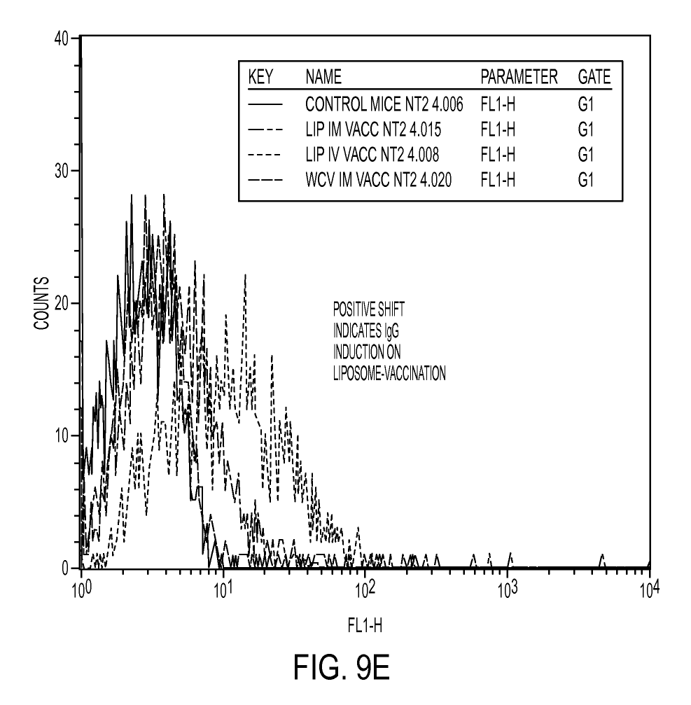

A control construct with no vaccine, a construct with 3T3-neu/GM and a pegylated liposome construct including NT2-GM-CSF were compared. FIG. 9 shows the Fluorescence Activated Cell Sorting (“FACS”) analysis of neu-specific IgG antibody (B-cell activation) in mouse serum using NT₂ cells and detected by secondary FITC-IgG₂ₐ antibody. Control group shows no B-cell activation (FIG. 9A) whereas the group that received intravenous and intramuscular liposome-vaccine injection show activation after 14 days (FIGS. 9B and C). Intramuscular injection of whole cell vaccine 3T3 neu-GM study which is used as positive control is also shown below in the panel (FIG. 9D).

### Example 10

### Liposome-Vaccine Treatment Protocol

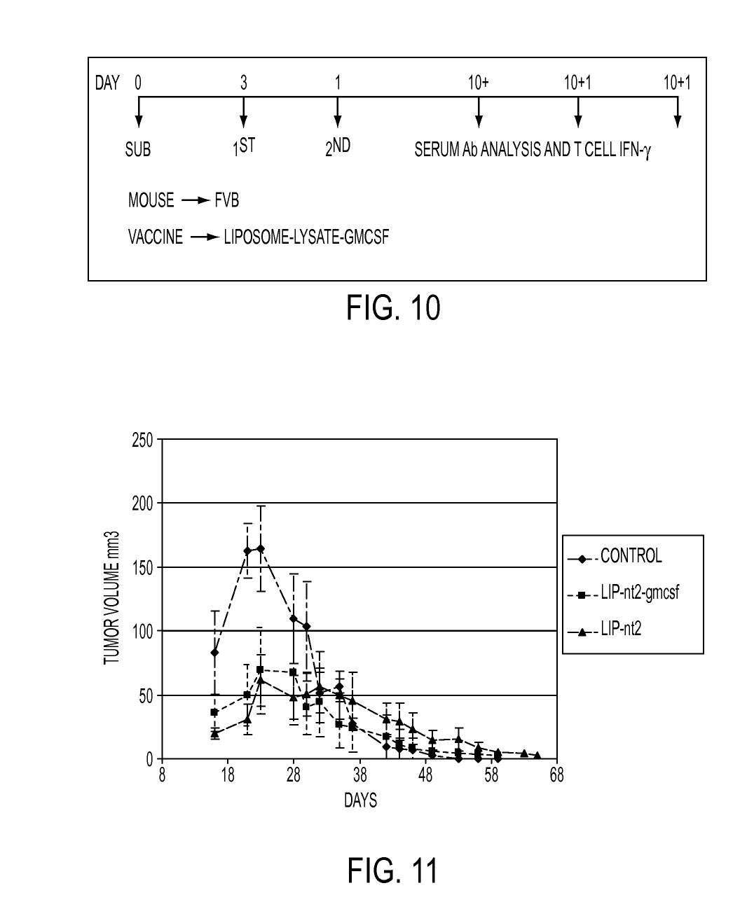

Two types of liposomal nanovaccines (LNVs) were examined: with and without GM-CSF. Subcutaneous injection of NT₂ mouse tumor cells into FVB mice on day 0 were made followed by intravenous injection of liposome-lysate vaccine on day 3 and day 10. Antibody in serum for B cell activity were analyzed using Fluorescence Activated Cell Sorting (“FACS”) on day 7, 10, 14 after treatment. FIG. 10 shows the timeline of liposome-vaccine treatment protocol.

### Example 11

### Tumor Growth Delay Analysis

Tumor growth delay was analyzed in FVB mice. FIG. 11 represents the tumor growth rate with and without liposome-vaccine after subcutaneous injection of 10⁶ NT₂ cells in FVB mice. The mouse tumor volume was monitored externally using a caliper after 2 weeks of tumor cells inoculation. The mice in the control group show maximum tumor volume of ˜180 mm³ at ˜3 weeks whereas the mice that received either the liposome-lysate vaccine with or without adjuvant (GM CSF) show a significant reduction in tumor size growth.

The control tumor regresses in these mice because they are not completely tolerant to tumor induction (i.e., they have an immune system that recognizes the tumor as foreign), efficacy in this model is demonstrated by the initial tumor growth relative to control. IV-administered LNVs are shown to lead to substantial tumor growth reduction.

### Example 12

### B-Cell Induced Humoral Ab Response

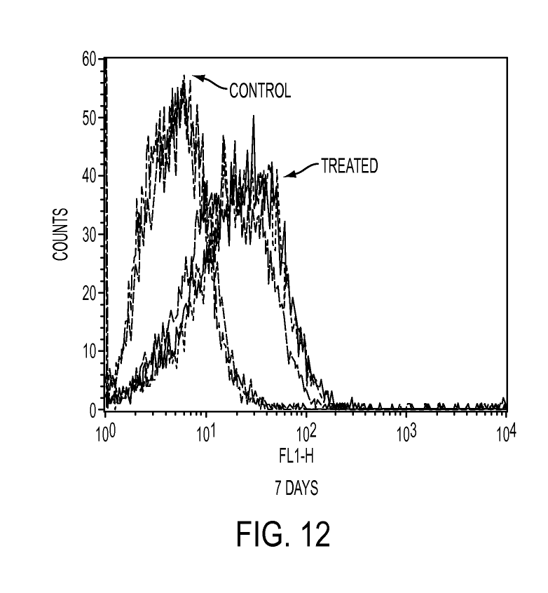

Using an antibody (Ab2) against anti-Neu antibodies, a B-cell induced humoral Ab response was measured 7 days after injection of the lip-NT₂-GM-CSF vaccine. FIG. 12 shows the histogram representation of FACS analysis of neu-specific IgG antibody (B-cell activation) in mouse serum using NT₂ cells and detected by secondary FITC-IgG₂ₐ antibody after 7 days of treatment with liposome-lysate-GMCSF. Treatment group shows positive shift compared to control group in FITC signal after day 7.

References cited herein are listed below for convenience and are hereby incorporated by reference in their entirety.
1. Banchereau J, Steinmann, R M. 1998, Dendritic cells and the control of immunity. Nature 392, 245-252.
2. Castile, et al., “Factors affecting the size distribution of liposomes produced by freeze-thaw extrusion,” International Journal of Pharmaceutics 188 (1999) 87-95.
3. Dijkstra C D. 1982, Characterization of nonlymphoid cells in rat spleen, with special reference to strongly Ia-positive branched cells in T-cell areas. J. Reticuloendothel Soc. September, 32(3), 167-78.
4. Emfietzoglou D, Kostarelos K, Papakostas A, Yang W H, Ballangrud A, Song H, Sgouros G. 2005, Liposome-mediated radiotherapeutics within a vascular tumor spheroids:comparative dosimetry study for various radionuclides, liposome systems, and a targeting antibody. J Nucl Med. January, 46 (1), 89-97.
5. Emfietzoglou D, Kostarelos K, Sgouros G. 2001, An analytic dosimetry study for the use of radionuclide-liposome conjugates in internal radiotherapy. J. Nucl. Med. Mar, 42 (3), 499-504.
6. Ercolini A M, Ladle B H, Manning E A, Pfannenstiel L W, Armstrong T D, Machiels J-P H, Bieler J G, Emens L A, Reilly R D, and Jaffee E M. 2005, Recruitment of latent pools of high-avidity CD8⁺ T cells to the antitumor immune response. JEM, 201 (10), 1591-1602.
7. Jones, et al., “Expression of dominant-negative ErbB2 in the mammary gland of transgenic mice reveals a role in lobuloalveolar development and lactation,” Oncogene (1999) 18, 3481-3490.
8. Kostarelos K, Emfietzoglou D, Papakostas A, Yang W H, Ballangrud A M, Sgouros G. 2005, Engineering lipid vesicles of enhanced intratumoral transport capabilities:correlating liposome characteristics with penetration into human prostate tumor spheroids. J. Liposome Res. 15 (1-2), 15-27.
9. Kostarelos K, Emfietzoglou D, Papakostas A, Yang W H, Ballangrud A, Sgouros G. 2004, Binding and interstitial penetration of liposomes within a vascular tumor spheroids. Int. J. Cancer. 20 November, 112 (4), 713-21.
10. Lasic, “Liposomes in Gene Delivery,” Biophysical Journal (April 1998) V. 74, 2138-2139
11. Machiels J P, Reilly R T, Emens L A, Ercolini A M, Lei R Y, Weintraub D, Okoye F I, Jaffee E M. 2001, Cyclophosphamide, doxorubicin, and paclitaxel enhance the antitumor immune response of granulocyte/macrophage-colony stimulating factor-secreting whole-cell vaccines in HER-2/neu tolerized mice. Cancer Res. 1 May, 61(9), 3689-97.
12. Murata S, Ladle B H, Kim P S, Lutz E R, Wolpoe M E, Ivie S E, Smith H M, Armstrong T D, Emens L A, Jaffee E M, Reily R T. 2006, OX40 Costimulation synergizes with GM-CSF whole-cell vaccination to overcome established CD8⁺ T cell tolerance to an endogenous tumor antigen. J. Immunol. 176, 974-983.
13. Reilly, R T., Gottlieb, M B C, Ercolini, A E, Machiels, J-P H, Kane, C E, Okoye, F I, Muller, W J, Dixon, K H, Jaffee, E M. 2000, Her-2/neu is a tumor rejection target in tolerized HER-2/neu transgenic mice. Cancer Res. 60, 3569-3576.
14. Slingluff C L Jr., Speiser D E. Progress and controversies in developing cancer vaccines. J Transl Med 2005; 3:18.
15. Sofou S, Thomas J L, Lin H, McDevitt M R, Scheinberg D A, Sgouros G. 2004, Engineered liposomes for potential alpha-particle therapy of metastatic cancer. J Nucl. Med. 1 February. 45(2), 253-260.
16. Song H, Shahverdi K, Fox J, Wang Y, Gimi B, Pomper M, Bhujwalla Z, Reilly R T, Sgouros G. 2004 A model of metastatic breast carcinoma for targeted alpha-emitter therapy modeling/dosimetry studies. Canc. Biother. Rad. 15 August, 19 (4), 518-518.
17. Song H, Shahverdi K, Fox J, Zhang Z, Reilly R T, Sgouros G. 2006, Targeting HER2/neu expressing micrometastases in HER2/neu transgenic mice using alpha-particle emitting monoclonal antibody [(BI)-B-213-7.16.4]. Canc. Biother. Rad. 41 August, 21 (4), 396-396.
18. Song H, Shahverdi K, Riley R T, Yoder B, Fox J, Qi Y, Wang Y, Brady E, Milenic D, Brechbiel M, Huso D, Pomper M, Tsui B, Sgouros G. 2004, A bone metastasis model for absorbed dose-response studies in targeted radionuclide therapy. Eur. J. Nuc. Med. Mol Imaging. 2 August, 31, S297-S297.
19. Stein H, Bonk A, Tolksdorf G, Lennert K Rodt H, Gerdes J. 1980, Immunohistologic analysis of the organization of normal lymphoid tissue and non-Hodgkin's lymphomas. J Histochem. Cytochem. 28 August, (8), 746-60.
20. van Broekhoven C L, Parish C R, Demangel C, Britton W J, Altin J G. Targeting dendritic cells with antigen-containing liposomes: a highly effective procedure for induction of antitumor immunity and for tumor immunotherapy. Cancer Res. 2004 Jun. 15; 64(12):4357-65.

1. A method for preventing, reducing or treating a condition comprising eliciting an immune response specific to said condition by delivering to the spleen of an individual a pegylated liposome construct, wherein said liposome has a diameter of greater than about 300 nm and comprises a therapeutic agent and an adjuvant for eliciting the immune response.

2. (canceled)

3. (canceled)

4. (canceled)

5. The method of claim 1, wherein the condition is a tumor cell isassociated with a cancer selected from the group consisting of carcinomas of the gastrointestinal or colorectal tract, liver, pancreas, kidney, bladder, prostate, endometrium, ovary, testes, melanoma, dysplastic oral mucosa, invasive oral cancers, small cell and non-small cell lung carcinomas, hormone-dependent breast cancers, hormone independent breast cancers, transitional and squamous cell cancers, neurological malignancies, osteosarcomas, soft tissue sarcomas, hemangioamas, endocrinological tumors, hematologic neoplasias, carcinomas in situ, hyperplastic lesions, adenomas, fibromas, histiocytosis, chronic inflammatory proliferative diseases, vascular proliferative disease, virus-induced proliferative diseases, leukemia, lymphoma, myeloproliferative disease, lymphoproliferative disease, neuroblastoma, glioma, and astrocytoma.

6. (canceled)

7. The method of claim 1, wherein the condition is breast cancer.

8. The method of claim 1, wherein the therapeutic agent is an immunogen.

9. The method of claim 1, wherein the condition is cancer and the therapeutic agent is a surface antigen specific to the tumor cell or a tumor-specific antigen, molecule, peptide or protein.

10. (canceled)

11. (canceled)

12. The method of claim 9, wherein the surface antigen is included in a concentrated mixture or in a cell lysate derived from the tumor cell.

13. The method of claim 1, wherein the therapeutic agent is tumor antigen selected from the group consisting of cancer-associated antigens belonging to gene products of mutated or recombined cellular genes, tumor virus antigens, overexpressed or tissue-specific differentiation antigens, and widely expressed antigens; or fragments or derivatives of any of the foregoing.

14. The method of claim 1, wherein the therapeutic agent is a tumor antigen selected from the group consisting of cyclin-dependent kinase 4 (CDK4), p151^{nk4b}, AFP, β-catenin, caspase 8, p53, p21^{Ras} mutations, Bcr-abl fusion product, MUM-I MUM-2, MUM-3, ELF2M, HSP70-2M, HST-2, KIAA0205, RAGE, myosin/m, 707-AP, CDC27/m, ETV6/AML, TEL/Aml1, Dekcain, LDLR/FUT, Pml-RARαTEL/AMLI, NY-ESO-I, members of the MAGE-family (MAGE-A1, MAGE-A2, MAGE-A3, MAGE-A4, MAGE-A6, MAGE-10, MAGE-12), BAGE, DAM-6, DAM-10, members of the GAGE-family (GAGE-I, GAGE-2, GAGE-3, GAGE-4, GAGE-5, GAGE-6, GAGE-7B, GAGE-8), NA-88A, CAG-3, RCC-associated antigen G250, human papilloma virus (HPV)-derived E6 E7 oncoproteins, Epstein Barr virus EBNA2-6, LMP-1, LMP-2, gp77, gp100, MART-1/Melan-A, p53, tyrosinase, tyrosinase-related protein (TRP-I and TPR-2), PSA, PSM, MCIR, ART4, CAMEL, CEA, CypB, HER2/neu, hTERT, hTRT, ICE, Muc1, Muc2, PRAME RU1, RU2, SART-I, SART-2, SART-3, and WT1.

15. The method of claim 1, wherein the therapeutic agent is a neu related protein, peptide or antigen.

16. The method of claim 1, wherein the therapeutic agent is a neu breast cancer antigen derived from a human tumor cell.

17. (canceled)

18. The method of claim 1, wherein the adjuvant is an activator of the immune system by stimulating receptors or pathways or both within cells of the immune system.

19. The method of claim 1, wherein the adjuvant is selected from the group consisting of unmethylated DNA comprising CpG dinucleotides (CpG motif); gel-like precipitates of aluminum hydroxide (alum); bacterial related proteins and products from the outer membrane of Gram-negative bacteria; synthetic lipopeptide derivatives; peptidoglycan; zymosan; heat shock proteins (HSP); dsRNA and synthetic derivatives thereof; polycationic peptides; taxol; fibronectin; flagellin; imidazoquinoline; cytokines with adjuvant activity; Tween 80 and Span 85 (sorbitan-trioleate) and QS-21, a more highly purified derivative of Quil A, non-ionic block polymers, saponins and derivatives thereof; polyphosphazene; N-(2-Deoxy-2-L-leucylamino-.beta.-D-glucopyranosyl)-N-octadecyldodecanoyl-amide hydroacetate (BAY R1005), 25-dihydroxyvitamin D3 (calcitriol); DHEA; murametide [MDP(Gln)-OMe]; murapalmitine; polymers of lactic and/or glycolic acid; polymethyl methacrylate; sorbitan trioleate; squalane; stearyl tyrosine; theramide, synthetic oligopeptides, CpG ODN with phosphorothioate (PTO) backbone (CpG PTO ODN) or phosphodiester (PO) backbone (CpG PO ODN): monophosphoryl lipid A (MPLA), lipopolvsaccharides (LPS), muramyl dipeptides and derivatives thereof; Pam.sub.3Cys; HSP 70; Poly I:poly C; poly-L-arginine; GM-CSF, interleukin-(IL-)2, IL-6, IL-7, IL-18, type I and II, interferons, interferon-gamma, TNF-alpha; MF59 consisting of squalene; Poloxamer 401, immunostimulatory fragments from saponins; and MHCII-presented peptides.

20. (canceled)

21. The method of claim 1, wherein the adjuvant is a cytokine or an agent that stimulates cytokine receptors.

22. The method of claim 1, wherein the adjuvant is GM-CSF.

23. The method of claim 1, wherein the liposome construct has a diameter of from about 300 to about 1000 nm, from about 400 to about 900 nm, from about 500 to about 800 nm, from about 600 to about 700 nm, from about 700 to about 800 nm, from about 600 to about 650 nm, from about 650 to about 700 nm, or of about 600, 650, 700, 750, or 800 nm, or a range within these sizes.

24. The method of claim 1, wherein the liposome has a diameter of about 650 nm.

25. The method of claim 1, wherein liposome construct comprises a polyethylene glycol (PEG) moiety with a molecular mass of 1000 to 10000 g/mol.

26. (canceled)

27. The method of claim 1, wherein the liposome construct is administered intravenously to the individual.

28. The method of claim 1, wherein the liposome construct is administered to a human in a dose of an effective amount of from about 1.0×10¹⁵ to about 1.0×10²⁰ liposomes/kilogram of body weight.

29. The method of claim 1, wherein the liposome construct is administered to a human in a dose of an effective amount of from about 4.7 to about 15 nanomoles of liposome concentration/kilogram of body weight.

30. The method of claim 1, wherein the liposome construct is administered to a human as a one-time dose or daily, weekly, every two weeks, or every month.

31. The method of claim 1, wherein the therapeutic agent is included in a cell lysate and provided in an amount from about 1 to 20 mg/ml per dose.

32. (canceled)

33. The method of claim 1, wherein the therapeutic agent is provided in a concentrated form/mixture in an amount from about 1.0 to 1000 mg/ml per dose.

34. The method of claim 1, wherein the adjuvant is provided in an amount from about 0.01 to 2.0 mg/ml per dose.

35. (canceled)

36. The method of claim 1, wherein the therapeutic agent is in an amount of from about 3 to 30% w/w of the liposome construct.

37. The method of claim 1, wherein the adjuvant is in an amount of from about 0.0029 to 0.29% w/w of the liposome construct.

38. The method of claim 1, wherein the liposome construct is administered in an effective amount to provide sufficient activation of the individual's immune system, generation of antibody or eradication of antigen expressing (Ag+) cells.

39. The method of claim 1, wherein the liposome construct causes an increase of the immune response at least by 5%.

40. (canceled)

41. The method of claim 40, wherein the antibody increase is by at least 5% to 100 fold.

42. (canceled)

43. The method of claim 1, wherein the liposome construct is administered to treat cancer and causes a cessation in tumor growth, a decrease of tumor size or growth delay or eradication of tumor cells.

44. (canceled)

45. The method of claim 1, wherein the administered liposome construct is formulated so that it localizes to the spleen rapidly and at very high concentration.

46. The method of claim 45, wherein the liposome construct is localized to the spleen within one hour after administration.

47. The method of claim 45, wherein the liposome construct is localized to the spleen in an amount of greater than 100% ID/gm. after administration.

48. The method of claim 1, wherein the administered liposome construct is formulated so that it is directed to the periarteriolar lymphoid sheath (PALS) contained within the white pulp (WP) region of the spleen.

49. The method of claim 1, wherein the administered liposome activates host adaptive immunity and recruits cytotoxic T lymphocytes (CTL) to eradicate the tumor cells.

50. The method of claim 1, wherein the administered liposome activates naïve T-lymphocytes, antigen-presenting cells (APCs) and interdigitating (reticulum) cells (IDCs) that are derived from circulating dendritic cells in the spleen.

51. The method of claim 1, wherein the administered liposome overcomes an individual's immune tolerance of the tumor cells.

52. A composition comprising a pegylated liposome construct formulated for delivery to the spleen of an individual, wherein said liposome construct has a diameter of greater than about 300 nm and includes a therapeutic agent and an adjuvant for eliciting the immune response in said individual for preventing, reducing or treating a condition.

53. (canceled)

54. (canceled)

55. (canceled)

56. (canceled)

57. (canceled)

58. The composition of claim 52, wherein the condition is cancer and the therapeutic agent is a surface antigen specific to the tumor cell or a tumor-specific antigen, molecule, peptide or protein.

59. (canceled)

60. (canceled)

61. The composition of claim 52, wherein the therapeutic agent is a neu related protein, peptide or antigen.

62. The composition of claim 52, wherein the therapeutic agent is a neu breast cancer antigen derived from a human tumor cell.

63. The composition of claim 52, wherein the adjuvant is an activator of the immune system by stimulating receptors or pathways or both within cells of the immune system.

64. The composition of claim 52, wherein the adjuvant is a cytokine or an agent that stimulates cytokine receptors.

65. The composition of claim 52, wherein the adjuvant is GM-CSF.

66. The composition of claim 52, wherein the liposome construct has a diameter of from about 300 to about 1000 nm.

67. The composition of claim 52, wherein the liposome has a diameter of about 650 nm.

68. The composition of claim 52, wherein liposome construct comprises a polyethylene glycol (PEG) moiety with a molecular mass of 1000 to 10000 g/mol.

69. (canceled)

70. The composition of claim 52, wherein the liposome construct is formulated as a single dose to be administered intravenously to an individual.

71. The composition of claim 52, wherein the liposome construct is formulated as a single dose comprising an effective amount of from about 1.0×10¹⁵ to about 1.0×10²⁰ liposomes.

72. The composition of claim 52, wherein the liposome construct is formulated as a single dose comprising an effective amount of from about 4.7 to about 15 nanomoles of liposome concentration

73. The composition of claim 52, wherein the therapeutic agent is included in a cell lysate and provided in an amount from about 1 to 20 mg/ml per dose.

74. (canceled)

75. The composition of claim 52, wherein the therapeutic agent is provided in a concentrated form/mixture in an amount from about 1.0 to 1000 mg/ml per dose.

76. The composition of claim 52, wherein the adjuvant is provided in an amount from about 0.01 to 2.0 mg/ml per dose.

77. (canceled)

78. The composition of claim 52, wherein the therapeutic agent is in an amount of from about 3 to 30% w/w of the liposome construct.

79. The composition of claim 52, wherein the adjuvant is in an amount of from about 0.0029 to 0.29% w/w of the liposome construct.
